(19) United States
(12) Patent Application Publication
(10) Pub. No.: US 2003/0202989 A1
(43) Pub. Date: Oct. 30, 2003

(54) USE OF TOXIN PEPTIDES AND/OR AFFINITY HANDLES FOR DELIVERING COMPOUNDS INTO CELLS

(76) Inventors: R. JOHN COLLIER, WELLESLEY, MA (US); STEVEN R. BLANKE, HOUSTON, TX (US); JILL C. MILNE, BROOKLINE, MA (US); ERICKA L. BENSON, KALAMAZOO, MI (US); JIMMY D. BALLARD, NORMAN, OK (US); MICHAEL N. STARNBACH, NEEDHAM, MA (US)

Correspondence Address: KRISTINA BIEKER BRADY, CLARK & ELBING, 101 FEDERAL STREET, BOSTON, MA, 02110, US

(21) Appl. No.: 09/077,439

(22) PCT Filed: Dec. 13, 1996
(86) PCT No.: PCT/US96/20463

(30) Foreign Application Priority Data
Dec. 13, 1995 (US) 60/008518
Jun. 7, 1996 (US) 60/019275

(51) Int. Cl. A61K 39/02; C12P 21/04; C12N 1/21; C07K 14/195; C07H 21/04; C12N 15/74

(52) U.S. Cl. 424/236.1; 514/12; 530/350; 435/69.7; 435/320.1; 435/252.3; 536/23.7

(57) ABSTRACT

A method and compositions for delivering a compound to the cytoplasm of a cell are disclosed. The compound to be delivered may be an antigenic compound, may be linked to a polycationic affinity handle, or both. In one of the methods disclosed, the B moiety of a toxin, such as the anthrax PA polypeptide, is also provided to enhance delivery of the compound to the cytoplasm of the cell.

This application claims priority from U.S. Ser. No. 60/008,518, filed Dec. 13, 1995 and U.S. Ser. No. 60/019,275, filed Jun. 7, 1996.

### STATEMENT AS TO FEDERALLY SPONSORED RESEARCH

Funding for the work described herein was provided by the National Institutes of Health Grants A1-22021, A1-22848, and A1-39558. The federal government has certain rights to the invention.

### BACKGROUND OF THE INVENTION

The invention relates generally to the delivery of compounds into living cells.

Many bacterial protein toxins enzymatically modify specific intracellular constituents of eukaryotic target cells. Mechanisms by which these enzymes traverse membrane barriers to contact their cytosolic substrates are being investigated because of their importance in a variety of biological contexts. Of particular interest is the development of such toxins into efficient and safe general protein-delivery systems.

Bacterial toxins frequently have two functionally distinct moieties, termed A and B. The A moiety contains the catalytic activity, while the B moiety possesses determinants needed for the cytoplasmic delivery of the A moieties into target cells. These delivery determinants include receptor binding activity, and often, but not always, membrane penetration activity. Many bacterial toxins, such as diphtheria toxin, contain both moieties within a single polypeptide. Anthrax toxin, by contrast, is a member of the so-called binary toxins, a class in which the A and B functions inhabit separate proteins. Although separate, the proteins having the A and B functions interact during the intoxication of cells. Anthrax toxin uses a single B moiety, protective antigen (PA; 83 kDa), for the delivery of two alternative A moieties, edema factor (EF; 89 kDa) and lethal factor (LF; 89 kDa) into the cytoplasm.

EF is an adenylate cyclase, whereas LF has an unknown activity that induces cytokine production (or cytolysis, at high concentrations) in macrophages. According to the current model of anthrax toxin action, PA first binds to a specific cell-surface receptor. The amino-terminal 20 kDa portion (PA20) is then removed by a cellular protease, leaving the carboxy-terminal 63 kDa bound to the receptor (PA63). This processing event is thought to expose a binding site on PA63 to which EF and LF bind competitively. The toxin-receptor complex is internalized by receptor-mediated endocytosis, and within the acidic environment of the endosome, PA63 mediates the release of EF and LF into the cytoplasm. PA63 has been shown to form ion-conductive channels in membranes under acidic conditions. A heptameric, ring-shaped form of PA63 identified recently may be relevant to these channels and to the translocation of EF and LF.

Sequence similarities between EF and LF within the amino-terminal 300 residues led to the hypothesis that this region contains the determinants required for binding to, and translocation by, PA. This proposal is supported by findings showing that a fusion protein between the amino-terminal 254 residues of LF (LFN) and the catalytic domain of exotoxin A was able to enter cells in a PA-dependent manner. In addition, fusion of LFn to either the amino-terminus of the carboxy-terminus or the enzymatic A chain of diphtheria toxin (DTA) enables DTA to be translocated into the cytoplasm of mammalian cells in the presence of PA.

There are two general classes of toxins which have been used for the introduction of heterologous proteins into cells. The pore forming toxins, such as alpha toxin and streptolysin O, act at the cell surface by permeabilizing membranes. Despite the widespread use of these toxins, they possess the disadvantage of inducing leakage of cellular contents into the external medium. A second class of toxins are those which bind to the cell surface and are then internalized. These toxins can be engineered as chimeras, substituting the receptor binding domain with another domain, such as an antibody, to change cell specificity. The majority of chimeric toxins constructed to date have utilized either diphtheria toxin or exotoxin A.

All viruses and some bacterial and protozoan pathogens have evolved the ability to survive and replicate within mammalian cells. Immune recognition of these cytoplasmic pathogens results from the cell-surface display of peptide antigens processed from pathogen-associated proteins. These peptides are presented in context with host class I molecules encoded by the major histocompatibility complex (MHC-I), and cytotoxic T-lymphocytes (CTL) are activated following recognition of the foreign peptide in complex with MHC-I. Activated CTLs lyse the infected cell, secrete cytokines, and then proliferate and differentiate. Each of these steps plays an important role in clearing the host of the pathogen. Lysis of the target cell deprives the organism of its replicative niche and exposes the pathogen to elements of the humoral immune system. Secretion of cytokines has many effects, including enhancement of local immune responses. Proliferation of the CTL clone results in expansion of one set of reactive CTL to effect clearance of the pathogen from other infected cells, while differentiation provides a set of long-lived memory cells available to respond more quickly and effectively to subsequent challenge. Vaccines that prime these memory cells provide protection to the host upon reexposure.

For a vaccine to mimic infection by cytoplasmic pathogens it must introduce the target antigen(s) into the cytosol of host cells in vivo. This has been accomplished by expressing heterologous antigens in live viral or bacterial vectors; by using adjuvants; or by delivery of DNA expression vectors (DNA vaccines) (Melief et al., Immun. Reviews 146:167-177, 1995; Traversari et al., Immunogenetics 35:145-52, 1992; White et al., Vaccine 13:1111-1122, 1995) subsequent challenge. Vaccines that prime these memory cells provide protection to the host upon reexposure. (Shen et al., Proc. of the Nat'l. Acad. of Sci. 92:3987-91, 1995).

### SUMMARY OF THE INVENTION

We have discovered that anthrax toxin B moieties may be used to deliver epitopes which, in turn, elicit an antibody response by the immune system. Furthermore, we have discovered that cationic tags, termed “polycationic affinity handles,” can substitute for toxin B moieties in mediating the entry of compounds, including antigenic epitopes, into cells. In both cases, the entry of the compounds is facilitated by the presence of PA.

When we fused the cytotoxic T-lymphocyte (CTL) epitope (LLO₉₁₋₉₉) from the intracellular pathogen Listeria monocytogenes to the nontoxic PA-binding domain of LF (LFn; 255 residues) we discovered that an antigenic response was provoked. Furthermore, a CTL response against LLO₉₁₋₉₉ was primed in BALB/c mice when this fusion protein was injected together with PA. Upon challenge with L. monocytogenes, mice vaccinated with LFn-LLO₉₁₋₉₉ and PA showed a reduction of colony forming units in spleen and liver, compared to control mice. These results indicate that anthrax toxin, and other toxin proteins having similar membrane translocation properties, may be useful as a general CTL-peptide delivery system for both research and medical applications. By utilizing nucleus acid in place of the antigenic compound, this delivery system may also be used to deliver covalently linked nucleic acid into the.

In the first aspect, the invention provides a method of introducing one or more antigen compounds into the intracellular region of a cell, including contacting the cell with a fusion molecule having LFn, or a polycationic affinity handle, or a toxin delivery system related to LFn/PA linked to the antigenic compound to be delivered to the cell.

In some embodiments, the antigenic compound or nucleic acid is covalently linked to LFn, or a fragment thereof. Preferably, the fall length LFn is used, as provided in the examples herein. However, the other toxin delivery and polycationic tag molecules described herein may be employed. In another preferred embodiment, the covalent linkage is at the N-terminus or the C-terminus of LFn.

In one embodiment, the method also includes contacting the cell with a B moiety of toxin (for example, anthrax PA, or clostridium perfringens with toxin B). In preferred embodiments, the B moiety is anthrax PA (83 kD) or the 63 kDa carboxy-terminal domain of anthrax PA (PA63).

The bond linking the toxin delivery moiety or the polycationic affinity handle to the compound may be a covalent bond, or in the case of negatively charged compounds, electrostatic attraction. In various preferred embodiments, the bond is a covalent bond, such as a peptide bond, an amide bond, a thioether bond, or a disulfide bond.

In yet other preferred embodiments of the method, the fusion molecule further includes a cleavage site between said compound and said toxin delivery moiety or polycationic affinity handle. In another preferred embodiment, the fusion molecule may include a spacer, such as a glycime spacer.

In another aspect of the invention, the compound being delivered to the cell is nucleic acid (encoding one or more protective antigen). The LFn, toxin delivery, and polycationic portions of fusions and methods described above for delivering antigens may be adapted for this purpose.

In another related aspect, the invention features a kit for introducing an antigen or antigen-encoding nucleic acid into the cytoplasm of a cell. The kit includes a fusion molecule including a first portion comprising a toxin moiety (e.g., a LFn portion) or a polycationic affinity handle, or a related toxin molecule linked to a second moiety comprising the antigen, antigens, or antigen-encoding compounds. In a preferred embodiment the kit is also includes anthrax PA. The first peptide may be joined to the second moiety by any of the bonds described herein. Where the first peptide is joined to a second moiety, which is a peptide by a peptide bond, preferably at least one of the amino acids of the second peptide is arginine or lysine.

In preferred embodiments of the fusion molecule, the second moieties are selected from the group consisting of: DNA, RNA, or antigen.

In a third aspect, the invention features a composition which is a mixture of a fusion molecule and the B moiety of a toxin (e.g., anthrax PA or PA63), wherein the fusion molecule includes at least a first peptide linked by a covalent bond to a second peptide. In a preferred embodiment of this aspect of the invention, the fusion includes a portion which is at least 3 amino acids, and at least 3 of the amino acids are selected from the group consisting of arginine, lysine, and histidine; or is LFn (or a fragment thereof); or is a polypeptide sequence from a toxin system related to the anthrax PA toxin system.

In the fourth aspect, the invention provides a method of introducing a molecule into the intracellular region of a cell, including contacting the cell with a molecule having a polycationic affinity handle linked to the compound to be delivered to the cell. In one embodiment of this aspect of the invention, the method also includes contacting the cell with a B moiety of a toxin (for example, anthrax PA, or clostridium perfringens with toxin B). In preferred embodiments, the B moiety is anthrax PA (83 kD) or the 63 kDa carboxy-terminal domain of anthrax PA (PA63).

In another preferred embodiment of this aspect of the invention, the polycationic affinity handle comprises a peptide of 2 to 250 amino acid residues, preferably a peptide of 2 to 16 amino acid residues. In a related preferred embodiment, at least two of the amino acids of polycationic affinity handle peptide are selected from the group consisting of: arginine, lysine, and histidine, and at least 10% of the amino acids comprising said peptide are selected from the group consisting of: arginine, lysine, and histidine. In even more preferred embodiments, the polycationic affinity handle includes at least three amino acids selected from the group consisting of arginine, lysine, and histidine. Most preferably, the handle includes at least 6 arginine residues, at least three lysine residues, or at least six histidine residues. In another preferred embodiment the polycationic affinity handle has a pKₐ between 6.5 and 12.5.

The bond linking the polycationic affinity handle to the compound (or compounds) to be delivered into the cell may be a covalent bond, or in the case of negatively charged compounds, electrostatic attraction. In various preferred embodiments, the bond is a covalent bond, such as a peptide bond, and amide bond, a thioether bond, or a disulfide bond.

In other preferred embodiments of the method, one or more compounds are selected from a group consisting of: protein toxin molecules, apoptosis inducing molecules, protein components of the signal transduction pathway, DNA, RNA, antigens, proteins for genetic complementation, an imunogenic antigen, therapeutic peptides, and therapeutic proteins. For example, a polycationic tag may have two antigenic compounds covalently linked to it.

In yet other preferred embodiments of the method, the fusion molecule further includes a cleavage site between the compound and the cationic affinity handle and/or a spacer, such as a glycine or serine spacer.

In another related aspect, the invention features a kit for introducing a compound into the cytoplasm of a cell, the kit includes a fusion molecule having a polycationic affinity handle linked to the compound to be introduced into the cell. In a preferred embodiment the kit also includes the B moiety of a toxin. For example, the kit may include a PA polypeptide, (e.g., PA63), and the affinity handle covalently linked to the compound, as described above or, where appropriate, may provide a handle suitable to be linked to the compound by electrostatic attractions.

In a sixth aspect, the invention provides a fusion molecule for delivery of one or more peptides to the cytoplasm of a cell, wherein the fusion molecule comprises at least the first compound linked by a covalent bond to a second compound, the second compound having at least two amino acids, at least two of the amino acids being selected from the group consisting of arginine, lysine, and histidine. The first compound may be joined to the second compound by any of the covalent bonds described herein. Where the first compound is joined to the second compound that is a peptide by a peptide bond, at least one of the amino acids of the second peptide is preferably arginine or lysine.

In preferred embodiments of the fusion molecule the first compound is a polypeptide and is selected from the group consisting of: protein toxin molecule, an apoptosis inducing molecule, a protein component of the signal transduction pathway, DNA, RNA, an MHC class I antigen, a protein for genetic complementation, an imunogenic antigen, a therapeutic peptide, and a therapeutic protein.

In a seventh aspect, the invention features a composition which is a mixture of a fusion molecule including a polycationic affinity handle and the B moiety of a toxin (e.g., anthrax PA), said fusion molecule comprising at least a first peptide linked by a covalent bond to a second peptide. In this aspect of the invention, the second peptide has at least 3 amino acids, and at least 3 of the amino acids are selected from the group consisting of arginine, lysine, and histidine. Furthermore, where the covalent bond is a peptide bond, at least one of said amino acids is arginine or lysine.

Definitions of Claim Terms

“B moiety” means a toxin moiety as described herein. For example, Anthrax PA or clostridium perfringens iota toxin B are examples of B moieties known in the art.

“PA” means a polypeptide having at least 60%, preferably 90%, of at least one of the biological activities of the anthrax PA polypeptide described herein. It is understood that homologs and analogs have the characteristics of the anthrax PA described herein and may be used in the methods of the invention.

“PA63” means the carboxy-terminal portion of the PA polypeptide described herein having at least 60% of at least one of the biological activities of the PA63 polypeptide described herein. Preferably, the PA63 is the 63kD carboxy-terminal fragment of the anthrax PA polypeptide.

“Polycationic affinity handle” means a cationic substrate capable of promoting entry of a compound into a living cell. Preferably, the cationic substrate is an amino acid sequence comprising amino acids including lysine, arginine, and histidine. The amino acid sequence may be between 2 and 250 amino acids long, so long as it has a sequence of between 2 and 20 amino acids which comprises the amino acids arginine, lysine, and/or histidine. Preferably, at least 80% of the 2-20 amino acid sequence is comprised of a combination of arginines, lysines, and/or histidines. Also preferably, the 2 to 250 amino acid sequence is comprised of amino acids at least 10% of which are selected from the group consisting of arginine, lysine and histidine. In preferred embodiments the pKA of the 2 to 20 amino acid sequence between 6 and 13.

“Introducing” means providing a means by which a compound provided to the extracellular region of a cell may by localized to the intracellular regions of a cell. Preferably, the compound is provided to the cytoplasm.

“Fusion Molecule” means a molecule which includes a compound to be delivered into the cell and a toxin delivery molecule (e.g., LFn) or a polycationic affinity handle.

“Linked” means placed in physical proximity by a covalent electrostatic bond.

“Fused” means any covalent chemical bond attaching the affinity handle to the compound to be transported into the cell. Where the compound is nucleic acid, fused also means colocalization between the affinity handle and the compound via electrostatic bonds. Preferably, the bond is a peptide bond, a disulfide, a thioether bond, a peptide-nucleic acid bond, or an amide bond.

“Mixture” means a composition of more than one substance. The mixture may be formulated, or example, for research, diagnostic, or therapeutic purposes using methods known in the art.

“Compound” means any substance which it is desirable to deliver into the intracellular region of a cell. The compound may be, for example, a therapeutic polypeptide, a cytotoxic polypeptide, DNA, RNA (e.g. antisense RNA for therapeutic purposes), or a small molecule, such as an antigenic peptide.

“Antigenic compounds” may be protein sequences, antigenic fragments, or antigenic non-polypeptide molecules (e.g., synthetic compounds). The polypeptide sequence may be from any origin, but preferably is derived from bacterial, viral, or tumor antigen polypeptides.

“Derived” sequences are those which are modified to incorporate or substitute sequences which have been modified to enhance antigenicity, solubility, stability, or codon usage of the encoding nucleic acid. Derivatives may be made using techniques known to one skilled in the art.

“Toxin delivery molecule related to the anthrax system (or LEn)” means a toxin delivery molecule known to facilitate translocation of covalently linked compounds across mammalian cell membranes.

### BRIEF DESCRIPTION OF THE DRAWING

FIG. 1A-FIG. 1D. CTL mediated lysis to LLO₉₁₋₉₉ peptide coated P815 cells. Female BALB/c mice were injected with either LFn-LLO₉₁₋₉₉ plus PA or LLO₉₁₋₉₉-LFN with or without PA.

FIG. 2. Efficiency of stimulation as a function of the LFN-LLO₉₁₋₉₉₉ concentration. BALB/c mice were injected with 6 pmol of PA mixed with either 3 pmol, 0.3 pmol, 0.03 pmol or 0.003 pmol LFn-LLO₉₁₋₉₉. After five days of stimulation of harvested splenocytes, in vitro, the cells were assayed for their ability to lyse ⁵¹Cr-labeled P815 cells coated ( ) or not coated ( ) with peptide.

FIG. 3. Protection against L. monocytogenes following immunization with LFn-LLO₉₁₋₉₉ plus PA. BALB/c mice (6 per group) were vaccinated with LFn-LLO₉₁₋₉₉ plus PA and challenged four weeks later with 2×LD₅₀ of L. monocytogenes i.v, livers and spleens were harvested, and the number of L. monocytogenes colony forming units per organ was determined. Significance was calculated by Wilcoxon's-Rank Sum analysis.

FIGS. 4A-4C are representations of various peptides fused to the amino-terminus of DTA. 4A shows a hexahistidine motif polypeptide; 4B shows basic, acidic, and neutral residues substituted for the hexahistidine motif of the fusion peptide; 4C shows different lengths of lysine fusion peptides fused to the amino terminus of DTA.

FIG. 5 is a graph illustrating PA-mediated protein synthesis inhibition of CHO-K1 cells by His-6-DTA.

FIG. 6 is a graph illustrating the effect of active site mutations in DTA on the cytotoxicity of His-6-DTA fusion proteins in the presence of 2.0×10⁻⁸M PA.

FIG. 7 is a graph illustrating the extent to which the amino acid composition of the amino-terminal fusion peptides affects the ability of DTA to inhibit protein synthesis in CHO-K1 cells in the presence of 2.0×10⁻⁸M PA.

FIG. 8 is a graph showing that the number of lysine residues in the amino-terminal fusion peptide affects the ability of DTA-fusion proteins to inhibit protein synthesis in the presence of 2.0×10⁻⁸M PA.

FIG. 9 is a graph showing that LFN blocks cytotoxicity of LFN-DTA, but not Lys-6-DTA.

FIG. 10 is a graph showing that the Lys-6 peptide does not effectively block the cytotoxicity of either Lys-6-DTA or LFN-DTA. The Lys-6-peptide having the sequence KKKKKKGSGCG (5×10⁻¹² to 5×10⁻⁴M) was added to CHOK1 cells in the presence of PA (2×10⁻⁸M) and either Lys-6-DTA (5×10⁻¹⁰M) or LFN-DTA (1×10⁻¹¹M).

FIG. 11 is a graph illustrating the PA-independent enhanced delivery of DTA having an affinity handle into the cell, relative to DTA lacking an affinity handle.

FIG. 12 is a graph illustrating delivery of P60 217-225 peptide.

FIGS. 13A and 13B are graphs illustrating delivery of two epitopes with a single injection.

FIGS. 14A and 14B are graphs illustrating vaccination with multiple epitopes.

FIG. 15 is a graph illustrating delivery of a disulfide linked compound (LLO₉₁₋₉₉).

FIG. 16 is a graph illustrating delivery of LCMU epitope NP 118-126.

FIG. 17 is a graph illustrating delivery of LCMV epitope NP 396-404.

### DETAILED DESCRIPTION

Abbreviations: DTA=catalytic domain of diphtheria toxin; EF=anthrax edema factor; LF=anthrax lethal factor; LFN=the PA binding domain of LF, comprising the amino-terminal 254 residues of full length LF; PA=anthrax protective antigen; PA63=the carboxy-terminal 63 kDa of anthrax protective antigen; TCA=trichloroacetic acid.

Overview

The efficient delivery of proteins, small peptides, and other compounds into the cytoplasm of eukaryotic cells has a number of important biomedical and research applications. These applications include therapy for certain genetic diseases by protein complementation (such as introduction of the wild-type CFTR protein in cystic fibrosis patients), antigen presentation to elicit specific MHC class I-restricted immune responses and clonal expansion of the relevant CD8+ cytoplasmic T lymphocytes, modulation of the activity of cytoplasmic target proteins, conditional expression of a protein's biological activity, and introduction of a protein which has been modified in vitro (e.g., phosphorylated, radio labeled, isoprenylated, epitope-tagged, or mutated). The applications may also include delivery of DNA (e.g. for gene therapy) or RNA (e.g. antisense RNA for therapy). It may also be desirable to deliver small molecules for research diagnostic or therapeutic purposes. We provide new methods and compounds for achieving the above goals.

Delivery of Antigens and Nucleic Acids Encoding Antigens.

We describe a novel strategy for introducing CTL epitopes into the cytosol of host cells, in vivo and in vitro, using an intra cellularly acting toxin, anthrax toxin.

Anthrax toxin is composed of three proteins that act in binary combinations to elicit two toxic effects, lethality and edema. Lethal factor (LF) and edema factor are intra cellularly acting proteins, both of which require protective antigen (PA) for translocation to the cytosol of eukaryotic cells. Initially, LF and EF competitively bind to proteolytically activated PA (PA₆₃) at the cell surface. The protein complex is endocytosed, and LF/EF is translocated to the cytosol following endosomal acidification. Recently, it was found that the amino terminal 254-amino acid domain of LF (LFn) directs interactions with PA. LFn appears to contain all the information necessary for PA binding and translocation, but is devoid of lethal activity. Further work has shown that heterologous proteins genetically fused to LFn are delivered to the cytosol of cultured mammalian cells in the presence of PA.

It occurred to us that CTL-reactive epitopes fused to LFn might be delivered to the cytosol and generate a CTL response in vivo. To test this hypothesis we initially chose a CTL epitope from the cytoplasmic bacterium Listeria monocytogenes. We observed a surprisingly strong antigenic response and believe that other antigenic fragments to which a mammalian immune response is desirable may be readily substituted by one skilled in the art to generate specific immunity. Indeed, below we show that additional Listeria, viral, and cancer epitopes may be used in the methods of the invention.

In the system employed in our experiments, delivery occurs in vivo and only in the presence of PA, suggesting that presentation depends on cytosolic delivery of the epitope and does not result from degradation of LFN-LLO₉₁₋₉₉ followed by external loading of MHC-I.

In using the toxin system, we avoid many of the complications associated with other epitope delivery systems. This system does not require the use of live or attenuated viruses or bacteria and also avoids the administration of adjuvants or the unintentional introduction of foreign DNA into animals. This system has potential for use in the treatment of a broad range of diseases in which CTL responses are required for protection. For example, CTL epitopes have been characterized for pathogenic viruses and bacteria. Each of these epitopes represents a candidate for LFN-PA mediated peptide vaccination against the corresponding microbial disease. In addition, several cancer-related CTL epitopes have recently been identified that may serve as the basis for development of an toxin-based anti-tumor vaccine.

Antigenic Peptides

Examples of antigenic polypeptides which may be employed in the invention follow. These are merely illustrative examples and are not meant to limit the invention.

Human Papillomavirus 16 peptides (e.g., antigens E6 and E7, E7 peptide 49-57 RAHYNIVTF); human P53 peptides (e.g., V10 peptide FYQLAKTCPV); human immunodeficiency virus peptides (e.g., gp 120, P18 peptide RIQRGPGRAFVTIGK); MUC-I human cancer antigen peptides; peptides from proteins of MAGE gene family (e.g., MAGE-1 SAYGEPRKL, MAGE-3 FLWGPRALV); peptides from the human tyrosinase protein (e.g., Tyr-A2-1 MLLAVLYCL, Try-A@-2 YMNGTMSQV); Listeriolysin-O peptides e.g., LLO₉₁₋₉₉ GYKDGNEYI); P60 peptides (e.g., P60217-225 KYGVSVQDI); MART-1 peptides (e.g., M-9 AAAAAGIGILTV, M10-3 EAAGIGILTV); BAGE-1 peptides (e.g., AARAVFLAL); P1A peptides (e.g., P815A35-43 LPYLGWLVF); Connexin gap junction derived peptides (e.g., Mut 1 FEQNTAQP, MUT 2 FEQNTAQA); peptides/proteins from any of the following pathogens: Cytomegalovirus, Hepatitis B, Human Herpes Virus 1-5, Rabies Virus, Meassles Virus, Mumps Virus, Rubella Virus, Shigella, Mycobacterium tuberculosis and avium, Salmonella typhi and typhimurium, HTLV-I,II, Varicella zoster, Variola, Polio, Yellow Fever, Encephalitis viruses, and Epstein-Barr virus.

Other Toxins as Delivery Tools

Any binary toxin that shares significant structure function similarities with anthrax toxin may be employed in the methods of the invention.

DNA Delivery

Given our findings regarding intracellular delivery, constructs involving fusions with nucleic acid-binding proteins, such as VirE2 from Agrobacterium rumefaciens may be employed in the methods of the invention. Such a fusion may be used to deliver nucleic acid into cells. This may be done using standard techniques known to one skilled in the art.

Polycatonic Affinity Handles

When expressed as recombinant proteins in E. coli, the affinity handle-DTA fusion proteins of the invention exhibit ADP-ribosyltransferase activity. Because DTA alone is not taanslocated into cells, and thus has no effect on cells (except at very high concentrations), cytotoxicity can be used as a measure of PA-mediated translocation of DTA into CHOK1 cells. Enhancement of translocation in the presence of PA may also be measured using cytotoxicity of these fusions to CHOK1 cells. Cytotoxicity of the polycationic affinity handle-DTA fusion protein was found to be due to protein synthesis inhibition using this DTA cytotoxicity assay. When mutations that attenuated the ADP-ribosylation activity of the DTA moiety were introduced into the affinity handle DTA fusion molecule, cytotoxicity of the resulting fusion molecule was dramatically reduced. Thus, the polycationic affinity handles increase delivery to the cytoplasm without themselves conferring toxicity. The presence of the PA polypeptide greatly enhances this phenomenon.

While any cationic peptide works to some extent, affinity handle fusion peptides containing lysine residues facilitated cell entry better than affinity handles comprising arginine or histidine residues. Peptides containing only acidic or neutral hydrophilic residues appeared to be non-functional substitutions for LFN for the purpose of PA-mediated delivery of compounds into cells. Therefore, the overall cationic nature of the affinity handles appears to be an essential characteristic required to coordinate PA-mediated delivery of compounds into the cytoplasm.

These results indicate that PA-mediated translocation of a heterologous compound does not require the presence of LFN. In addition, affinity handles may enhance translocation of a compound even in the absence of PA. The affinity handles of the invention may be physically linked to a heterologous compound and used in combination with PA to create an efficient, non-toxic, heterologous compound delivery system. The physical linkage may be covalent or, in the case translocation of nucleic acids, may be electrostatic.

Theory

While the mechanism by which the affinity handles of the invention act has not been determined, one possible theory to explain the surprising results disclosed herein is as follows. Previous evidence has suggested that A-B toxins internalized by receptor mediated endocytosis requires direct molecular contact between the A moiety of the toxin and the B moiety of the toxin. For DT and a number of other toxins, the A and B moieties are covalently linked, while for binary toxins such as anthrax, there are high-affinity binding interactions between the A and B moieties. The experiments disclosed herein suggest that anthrax PA can facilitate translocation of a heterologous protein or other compound that binds either at an alternative site on the PA-oligomer, or to a distinct cell surface component that is cointernalized with receptor-bound PA during endocytosis.

The theory that Lys-6-DTA binds to a cell surface component distinct from receptor-bound PA63 suggests a possible explanation of how the cationic affinity handle may promote delivery of DTA to the cytoplasm. The polybasic peptide may have binding affinity for a surface component other than PA63, such as a protein with an exposed acidic domain. Candidates for such a surface component include the acidic glycoprotein, found to be a major class of membrane-protein constituents of CHOK1 cells (Raab, et al., 1990, J. Cell. Physiol. 144(1):52-61; Raab et al., 1986, Exp. Cell Res. 165(1):92-106), as well as acidic phospholipids on the plasma membrane. Numerous studies with both proteins and synthetic peptides have established the electrostatic interaction of basic amino acid side chains with acidic phospholipid head groups as an important mechanism of protein-lipid interactions (Gennis, R. B., 1989, Biomembranes: Molecular Structure and Function, pp. 86, 87, 196, 197, Springer-Verlag N.Y., Inc.). The myelin basic protein is the prototype of membrane proteins anchored primarily by the electrostatic interactions of basic residues with acidic phospholipids. Phospholipase C, protein kinase C, myristoylated alanine rich C kinase substrate (“MARCKS”), pp60v-src (the v-src oncogene of the Rous sarcoma virus), and lactoferrin all contain clusters of basic residues that have been shown to be specifically important for membrane interactions (Buser et al., 1995, Mol. Membrane Biol. 12(1):69-75). Studies with model peptides that mimicked the membrane binding regions of MARCKS and pp60v-src showed that each basic residue in the peptide binds independently to an acidic phospholipid, contributing microscopic binding energy congruent to 1 kcal/mole.

The discovery of heptameric PA structures linked to both channel formation and cytoplasmic delivery of LF and EF makes it attractive to postulate that the pore formed by the PA-oligomer may provide a conduit for protein translocation across the membrane. Alternatively, PA may liberate Lys-6-DTA by destabilizing the endosome, and inducing rupture of vesicles within which both components are endocytosed.

Regardless of the mechanism by which PA and Lys-6-DTA are co-internalized into the same endosome, these results do not appear to be particular to the specific amino acid sequence of the affinity handle or nature of the compound to be transported. Accordingly, these results define the elements of a general translocation system.

Advantage of PA-mediated Delivery of Fusion Molecules

In contrast to existing toxin chimera delivery systems, the anthrax toxin system of the invention eliminates the need to generate fusion proteins with a toxin B moiety. This, in turn, alleviates problems associated with incorrect folding of lengthy fusion proteins, leading to potential conformational inactivation of the B moiety and/or the target compound being delivered. Furthermore, substituting small cationic fusion peptides for LFN may reduce the possibility of steric interference with the biological activity of the translocated protein. In addition, the short, cationic peptide affinity handles can serve a dual purpose as affinity handle tags to expedite purification of the fusion proteins. Such techniques are well known in the art for the purification of compounds having a histidine-rich handle (i.e., via Ni chelate chromatography); we describe herein a procedure for purification of compounds having a lysine-rich handle.

Cationic Affinity Handles

The preferred cationic affinity handle is a peptide containing multiple lysine, histidine, and/or arginine residues. Our experiments have shown that 3-10 successive lysine residues work well in this system, with a greater number of residues facilitating better delivery of heterologous proteins. The pKa range attained using lysine, histidine, or arginine should be between 6-13 for the region of the handle which is rich in one or more of the following: lysine, arginine, or histidine. In addition to peptide bonds, it is possible that disulfide, thioether, or amide bonds can be used to attach the polycationic affinity handles to the heterologous protein or other compound. Furthermore, electrostatic attraction may be used to link the affinity handle to nucleic acids. We believe that neutral residues interspersed within the basic residues constituting the cationic affinity handle will not prevent enhanced delivery of the compounds into the cells.

Sources of PA

PA may be purified, for example, from the Sterne strain of Bacillus anthracis or synthesized by other known means. In Bacillus anthracis, the gene for PA is located on a plasmid referred to as pXO1 (Milne et al., 1994, J. of Biol. Chem. 269(32):20607-20612). PA63 can be substituted for full-length PA. This is the preferred approach where the target cell lacks the protein required to cleave full length PA into PA63. The PA63 fragment may be purified from trypsin-treated PA by anion exchange chromatography (Milne et al., 1994, supra). PA encoding gene has been cloned and sequenced (Vodkin, et al., 1983, Cell 34:693-697) and may be used to obtain purified PA polypeptide.

Compounds for Delivery to Cells.

The PA-mediated delivery system provided may be used to deliver a variety of different compounds to the cell. The method merely requires that a cationic affinity handle be linked to the compound to create the affinity handle-compound fusion molecule. This may be done using covalent bonds or, in the case of negatively charged compounds such as nucleic acids, electrostatic bonds. The fusion molecule may then be provided to the target cell before, after, or simultaneously with an amount of PA sufficient to allow delivery of the compound into the cell.

Cleavable Affinity Handles

The affinity handle may be linked to the compound by a sequence or other substrate known to be cleaved on the interior of the cell. Use of a cleavable spacer to link the affinity handle may be desirable where the handle is large or the handle is observed to otherwise interfere with the activity of the compound. Proteolytically cleaved polypeptide sequences are one example of the types of sequences which may be employed for this purpose.

Glycine Spacers

A spacer length of approximately seven residues is well tolerated in the delivery system described herein. Other lengths of spacers should also work well, and can be tested in the system provided (e.g., between 2 and 100). The spacer must be long enough such that the polycationic residues in the affinity handle are not satyrically blocked thereby preventing cell association/binding. Glycine residues are favored in a spacer, because they tend not to form significant secondary structural elements and thus may lend flexibility to the spacer, but other amino acids may be employed.

Bonds for Attaching Affinity Handles

The types of bonds used to attach the affinity handle to the compound of interest may be peptide, disulfide, thioether, amide bonds or peptide-nucleic acid bond (e.g., interribose linkages). Peptide bonds between the affinity handle and a heterologous protein may be constructed by genetically fusing the coding sequence for the affinity handle in frame to that of the protein to be targeted. Disulfide bonds may be constructed between a cysteine in the affinity handle and one in the protein to be targeted. The cysteines may be engineered into the coding sequence of the affinity handle or the protein to be targeted, if they do not already exist. Bond formation may then be carried out by solution oxidation. Thioether bonds may be constructed between a cysteine residue and an aliphatic carbon having a strong leaving group such as a halogen. Amide bonds would be formed between any carbonyl compound containing a strong leaving group and a compound containing a primary, secondary, or tertiary amine. A lysine residue would be a good example of a compound containing a primary amine.

In addition to the above, where nucleic acid delivery (or other highly negatively charged compounds) is desirable, electrostatic attractions between the nucleic acid and the affinity handle may be employed to join the handle to the compound. Alternatively, peptide-nucleic acid linkages may be employed (see e.g., Nielsen, P. E. et al., Trends in Biotechnology 11: 384-386 (1993); Agrawals and Iyer, R P, Current Opinion in Biotechnology 6: 12-19 (1995)).

Target Cell Types

While the invention is not limited by cell type, for PA-dependent methods the cell types targeted must express a functional PA receptor. To date all cell types tested have been able to bind PA (Leppla, S. H. review: Leppla, S. H. 1991. The Anthrax Toxin Complex in (J. E. Alouf, J. H. Freer, eds. Sourcebook of Bacterial Protein Toxins, Academic Press, London).

The following examples are provided to illustrate and not to limit the invention.

### EXAMPLES

### Example 1

### Materials and Methods

Cell Culture

The CHO-K1 cell line was obtained from the American Type Culture Collection (American Type Culture Collection, Bethesda, Md., ATCC CCL 61). Cells were grown in Ham's F-12 medium supplemented with 10% calf serum, 500 units/mL penicillin G, and 500 units/mL streptomycin sulfate (Life Technologies, Inc., Grand Island, N.Y.). Cell cultures were maintained as monolayers and grown in a humidified atmosphere of 5% CO₂.

Construction, Expression, and Purification of the DTA Fusion Proteins

Standard Protocols were used for all genetic manipulations (e.g., Ausubel et al., 1987, Current Protocols in Mol. Biol., John Wiley and Sons, New York). An entirely synthetic gene encoding DTA plus a 17-residue amino-terminal polyhistidine fusion peptide was used as the starting substrate to generate the constructs described in these investigations. The polybasic-DTA fusion proteins were generated by PCR reactions with primers containing the desired sequence and designed for annealing to the amino-terminus of the synthetic gene (Ausubel et al., 1987, Current Protocols in Mol. Biol., John Wiley and Sons, New York, Supplement 20). All of the constructs were cloned into the E. coli expression vector pET15b, replacing the NcoI-BamnHI fragment (Novagen Inc., Madison, Wis.). The ligation reactions were transformed into E. coli XL1-Blue (Stratagene, La Jolla, Calif.). The plasmid DNA was amplified, purified, and screened for the presence of the appropriate sequence (Ausubel et al., 1993, Current Protocols in Mol. Biol. John Wiley and Sons, New York). Those gene constructions confirmed as possessing the correct sequences were then transformed into the E. coli expression host BL21(DE3) (Studier and Moffatt, 1986, 1986, J. of Mol. Biol. 189:113-130).

The recombinant proteins expressed in pET15b are produced with an amino-terminal hexa-histidine tag, allowing the proteins to be purified by affinity chromatography on a Ni²⁺-charged column (Blanke et al., 1994, Biochemistry 33:5155-5161). Briefly, cultures of BL21(DE3)/pET15b-peptide-DTA were grown in Luria broth-ampicillin (100 μg/mL) to an OD600 nm of 0.6-1.0, and protein expression was induced by addition of 1 mM IPTG for approximately 4 h. Cells were lysed by sonication, cleared by centrifugation, and loaded onto a Ni²⁺-charged column (Blanke et al., 1994, supra). The chromatography step was performed according to the methods determined previously for the purification of His-6-DTA (Blanke et al., 1994, supra). The Qiagen system was used for the purification of His-6-DTA (Blanke et al., 1994, supra). All buffers and resins were as specified by the manufacturer. The column was washed and the protein eluted with imidazole. The eluted protein was desalted and further purified by anion-exchange chromatography (MonoQ™ column on a fast-protein liquid chromatography system; Pharmacia). Approximately 10 mg of purified protein was obtained from one liter of culture.

NAD:EF-2 ADP-Ribosyltransferase Assay

The NAD:EF-2 ADP-ribosyltransferase activity assay measures the initial rates of incorporation of the ADP-ribose moiety of [³²P]-NAD into the trichloroacetic acid (TCA)-precipitable EF-2 fraction of the reaction mixture. The assay was performed essentially as described (Blanke et al., 1994, supra), with initial rates determined by the collection of three linear time points in duplicate. Reaction mixtures contained 50 mM Tris-HCl, pH 8.0, 1 mM EDTA, 10 mM DTT, 50 μg BSA mL-1, 50 μM NAD, 0.5 μM EF-2, and enzyme. The reactions were incubated at 25° C. and aliquots were removed from duplicate samples at 2, 3, and 4 minutes and pipetted directly onto 3 MM filter paper (Whatman, Hillsboro, Oreg.). The filter pads were placed immediately into ice-cold 5% TCA, and washed 3-5 times for 15 minutes by gentle agitation on a platform rocker until no counts could be detected in the discarded wash solutions. The filter pads were then washed twice for 5 minutes in ice-cold methanol, dried, and counted with 3 mL of Beckman Ready Safe™ Liquid Scintillation Cocktail (Beckman, Columbia, Md.) in 1209 Rackbeta™ scintillation counter (LKB, Piscataway, N.J.). Initial rates were calculated based on the increase in counts (minus background) over 5 minutes with less than 10% of the reactants having been utilized.

Protein Synthesis Inhibition Assay

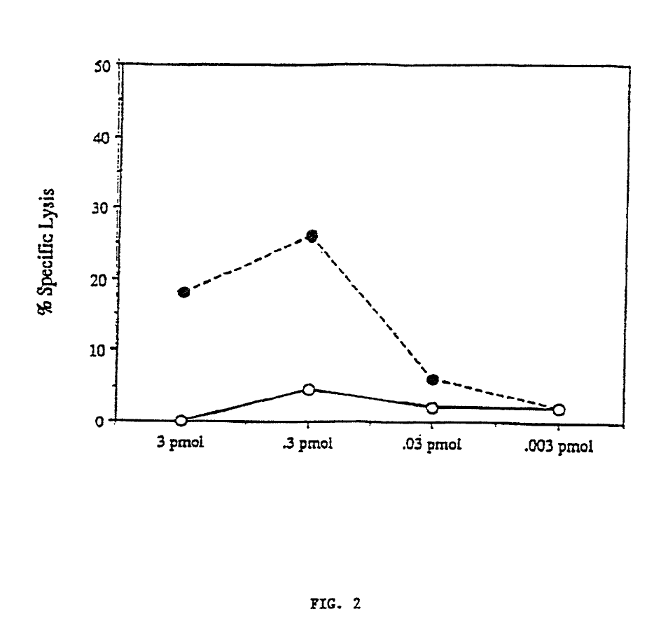

CHO-K1 cells were plated at a density of 4×10⁴ cells per well, in Costar 96-well cluster plates approximately 18 h prior to the start of an experiment (Costar Inc., Cambridge, Mass.). PA (2×10⁻⁸ hs M) and fusion proteins (concentrations indicated in FIGS. 2-7) were added to cells in Hams' F-12 medium. After 24 hours at 37° C., the medium was removed, the cells were washed with PBS (Gibco BRL, Grand Island, N.Y.), and L-leucine-deficient medium (Gibco BRL) supplemented with L-[3,4,5-³H]-leucine (1 μCi/mL; Dupont NEN, Boston, Mass.) was added. After 1 hour, the cells were washed with ice-cold PBS followed by ice-cold TCA (10%). Protein synthesis was measured by incorporation of radioactivity into acid insoluble material and expressed as the percentage of incorporation by unintoxicated control cells. All assays were performed in duplicate. Variations of this assay are indicated in FIGS. 2-7.

Splenocyte Harvesting

Mouse splenocytes were harvested and CTL stimulated as described (Starbach et al., J. of Immun. 153:1603, 1994) with the following modifications. Spleen cells from immunized and control mice were isolated and washed once in RP-10. Cells used as simulators were naive, irradiated (2000 rad), syngeneic splenocytes treated with 10 μM sterile LLO₉₁₋₉₉ peptide. The stimulator cells were incubated 1 h in the presence of peptide and then washed once in RP-10. Cultures contained 3×10⁷ stimulator cells and 3×10⁷ splenocytes from either immunized or control mice. These were incubated upright in T-75 flask at 37° C. in 70% CO₂ in a total volume of 20 ml RP-10.

### Example 2

### Vaccination with L. Monolytogenes LLO₉₁₋₉₉ Antigen

L. monocylogenes is a facultative intracellular bacterial pathogen that survives within the cytosol of macrophages. After phagocytosis, listeriolysin-O (LLO) lyses the phagosomal membrane allowing the bacterium to escape to the cytosol. LLO is proteolytically processed by host cells, generating peptides that are presented at the cell surface in context with MHC-I. Processing of LLO results in the presentation of a nonameric peptide LLO₉₁₋₉₉ (GYKDGNEYI), recognized by H-2 K^{d}-restricted CTL (Villanueva et al., J. of Immun. 155:5227-5233, 1995). Adoptive transfer studies have shown that CTL specific for LLO₉₁₋₉₉ are sufficient for protection against L. monocytogenes, suggesting LLO₁₋₉₉ as a vaccine candidate (Harty et al., J. of Exp. Med. 175:1531-8, 1992).

A DNA sequence encoding LLO₉₁-99 was genetically fused to the 5′ or the 3′ end of the gene fragment encoding LFN. The fusions, LFN-LLO₉₁₋₉₉ and LLO₉₁₋₉₉-LFN, were cloned into the expression plasmid pET-15b, and the recombinant proteins were expressed in Escherichia coli BL-21 and purified. PA was isolated from the supernatant of cultures of an attenuated strain of Bacillus anthracis according to an established protocol (Leppla, Adv. in Cyc. Nuc. & Prot. Phosph. Res. 17:189-98, 1984).

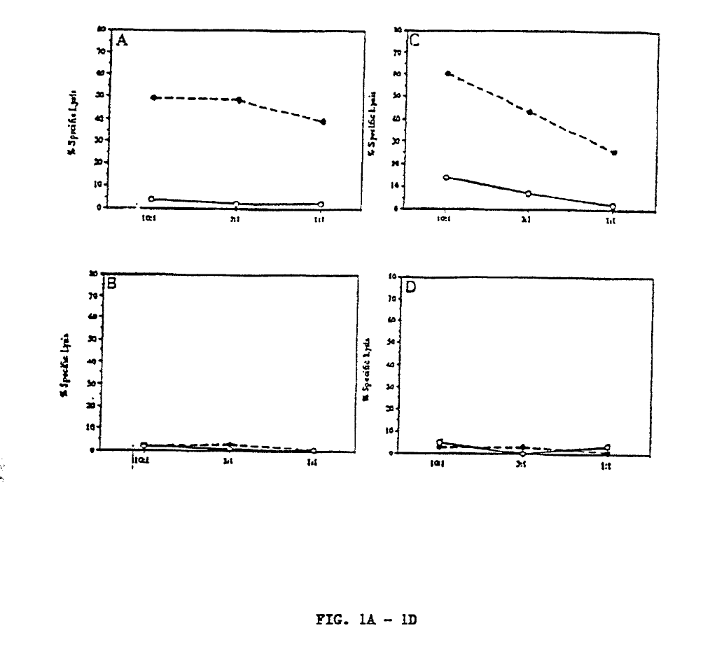

BALB/c mice (5 per group) were injected intraperitoneally with 30 pmol of either fusion protein, LFN-LLO₉₁₋₉₉ or LLO₉₁₋₉₉-LFN, plus 6 pmol of PA. Control groups of mice were injected with LFN-LLO₉₁₋₉₉ alone, LLO₉₁₋₉₉-LFN alone, PA alone, LLO₉₁₋₉₉ alone, or PA plus LLO₉₁₋₉₉. Fourteen days following injection the animals were sacrificed, and 3×10⁷ splenocytes were stimulated on syngeneic spleen cells coated with the LLO₉₁₋₉₉ peptide. After 5 days of stimulation in vitro, the cells were assayed for the ability to lyse an H-2^{d}mastocytoma, P815, coated with LLO₉₁₋₉₉. As shown in FIG. 1, lysis of peptide-coated P815 was substantially higher than lysis of P815 cells alone, indicating that the mice had mounted an LLO₉₁₋₉₉-specific CTL response. Priming occurred regardless of whether LLO₉₁₋₉₉ was fused to the carboxy or the amino terminus (FIG. 1). None of the controls stimulated a LLO₉₁₋₉₉ specific CTL response, indicating that this is an LFN-mediated, PA-dependent event.

The efficiency of this delivery system was examined by injecting a fixed amount of PA together with decreasing amounts of LFN-LLO₉₁₋₉₉. Mice were injected with 6 pmol of PA mixed with either 3 pmol, 0.3 pmol, 0.03 pmol, or 0.003 pmol of LFN-LLO₉₁₋₉₉. Splenocytes were harvested after 14 days and assayed for CTL activity after 5 days of stimulation in vitro. As shown in FIG. 2, priming was achieved with as little as 0.3 pmol of LFN-LLO₉₁₋₉₉.

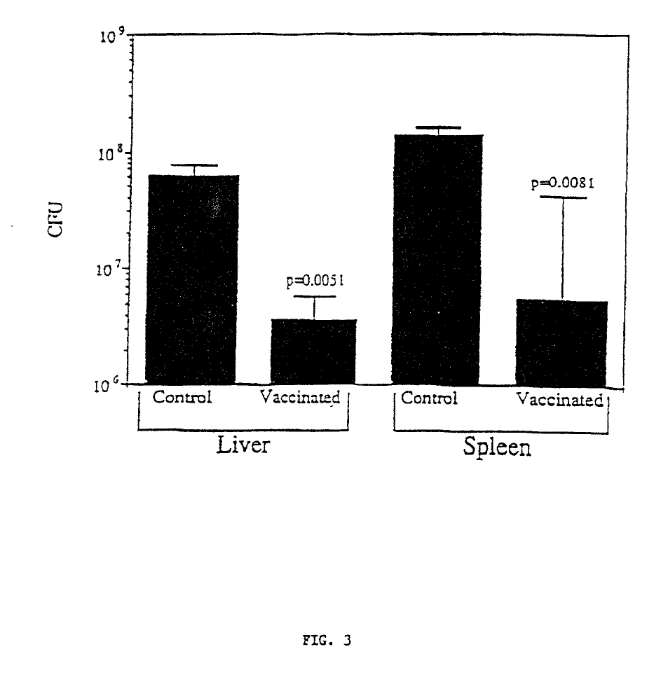

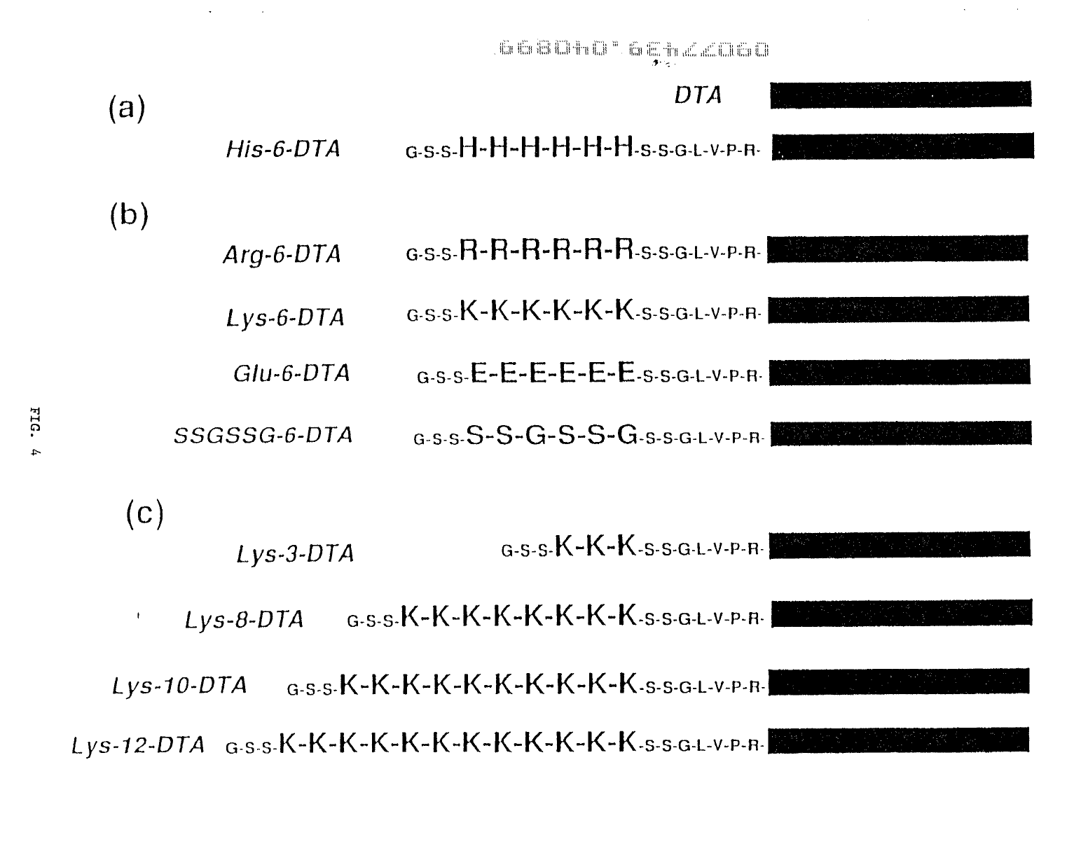

Experiments were then performed to determine if mice vaccinated with the LFN-LLO₉₁₋₉₉ fusion protein are protected against a challenge with Listeria monocytogenes. BALB/c mice were immunized with 30 pmol LFN-LLO₉₁₋₉₉ plus 6 pmol PA as in the previous experiments. Four weeks after immunization the mice were challenged intravenously with 2×LD₅₀ (11) (1×10⁴ colony forming units) of L. monocytogenes. Forty-eight hours after infection the mice were sacrificed, and spleens and livers were harvested. As shown in FIG. 3, significantly lower number of colony forming units were present in these organs in vaccinated mice compared with control mice (PBS alone). The vaccinated group showed an average of 30-fold fewer bacteria in the liver and an average of 20-fold fewer bacteria in the spleen.

### Example 3

### Delivery of Another Epitope from Listeria monoaytogenes

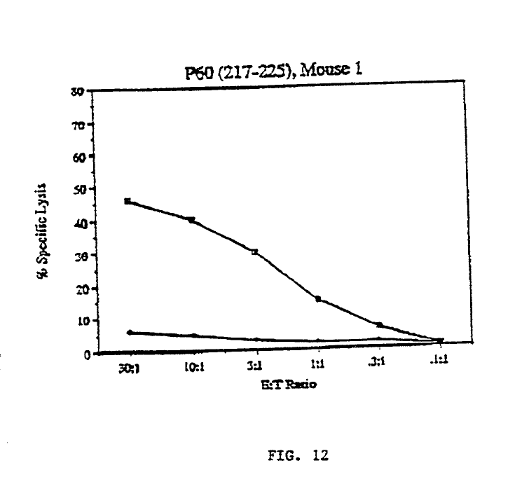

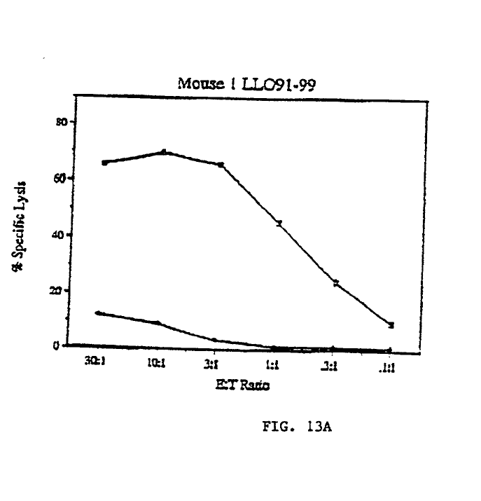

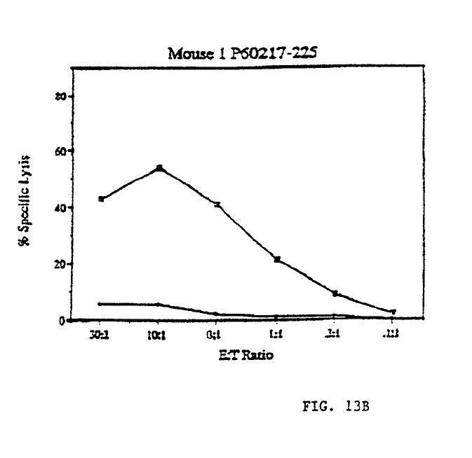

As described above, we have demonstrated delivery of the CTL epitope LLO₉₁₋₉₉ derived from listeriolysin O using the LFn-PA system. The epitope can be delivered fused to either end of LFn and the CTL response provides protection from challenge with Listeria monocytogenes. In addition to the above data, we have obtained similar results with another known CTL epitope from L. monocytogenes. This epitope, P60₂₁₇₋₂₂₅, appears to be efficiently delivered by LFn-PA and, as with the other epitope delivery, is LFn mediated and PA dependent. The results are shown in FIG. 12.

### Example 4

### Delivery of Epitopes From a Pathogenic Virus, Lymphocytic Choriomeningitis Virus (LCMV), to Two Haplotypes of Mice

We have found that the LFn-PA delivery system may be used to deliver 2 CTL epitopes from LCMV. Both epitopes tested are derived from the nucleocapsid protein of the virus and one is of the H2^{b} haplotype while the other is H2^{d}. The delivery of these two different epitopes demonstrates that this system works in more than one haplotype of mice. The data is shown in FIG. 17.

### Example 5

### Delivery of Two Cancer Protection Epitopes

Two epitopes used in cancer models have been delivered. The first epitope is derived from ovalbumin, OVA₂₅₇₋₂₆₅. This epitope is presented in a tumor cell line, EG7, that has been transformed to express ovalbumin. OVA₂₅₇₋₂₆₅ is not necessarily a tumor derived antigen, but provides a very convenient approach for assaying the vaccinating capacity of the anthrax toxin system against a solid tumor. The second epitope is P815A and is derived from the mouse mastocytoma P815.

### Example 6

### Delivery of Two Epitopes Either as Part of the Same Genetic Fusion, or on Separate LFn Molecules in a Single Injection

In this example we show that we can generate a CTL response to more than one epitope with a single injection. We have accomplished this by making fusions that express both epitopes on the same protein and, alternatively, by injecting a mixture of epitopes separately fused to LFn.

Mice injected with a mixture of LFn fused to either P60₂₁₇₋₂₂₅ or LLO₉₁₋₉₉ mount a strong CTL response to each of the epitopes.

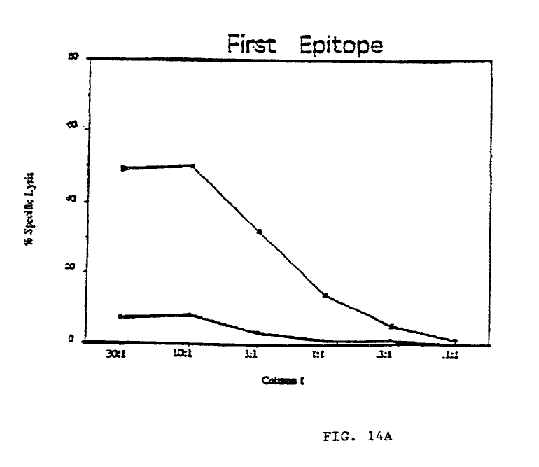

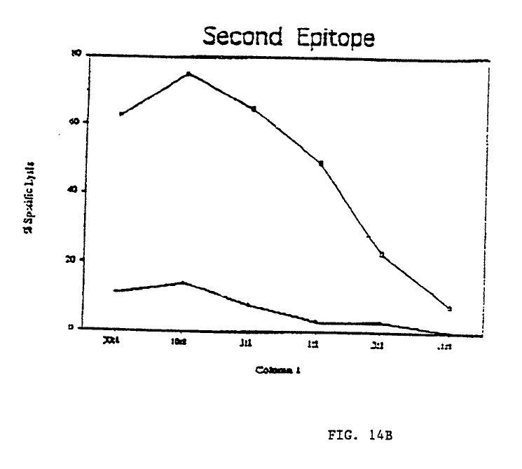

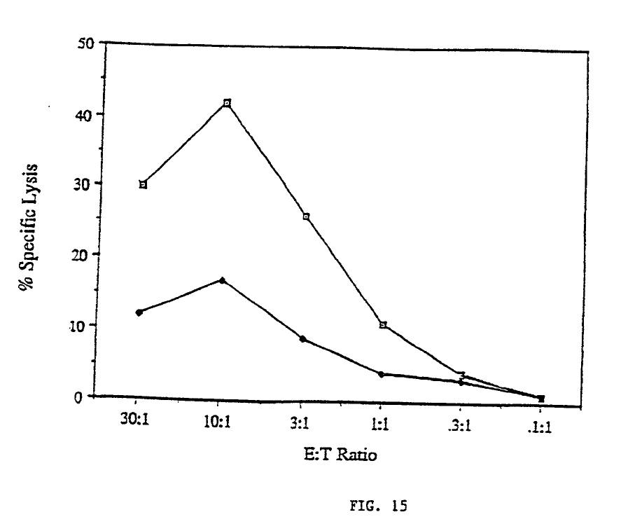

To test the efficacy of administering multiple epitopes on one fusion a LFn construct was made that expresses LFn fused to LLO₉₁₋₉₉ and the H2^{d} epitope of LCMV. Mice injected with this fusion mount a strong CTL response to both epitopes. These results are illustrated in FIG. 13 and FIG. 14.

Given these results, we believe this system can be used to protect against more than one infectious agent using a single injection.

### Example 7

### Delivery of an Epitope Fused to LFn via a Disulfide Bond

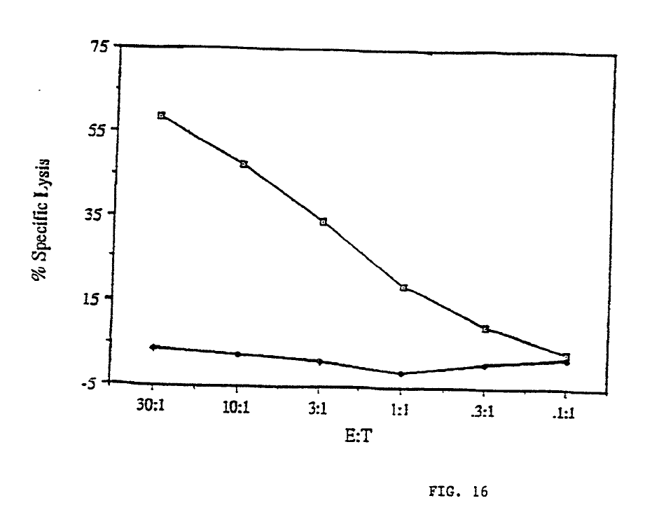

As an alternative approach for delivery of CTL epitopes with the LFn-PA system, we have disulfide linked LLO₉₁₋₉₉ to LFn and assayed for a CTL response to this epitope. A synthetic form of LLO₉₁₋₉₉ was made to contain a single cysteine in addition to other sequence. This peptide was then oxidized to a mutant of LFn that contains a single cysteine. Mice injected with this heterodimer and PA mount a LLO₉₁₋₉₉-specific CTL response. FIG. 15 illustrates these results. This approach provides a method for delivering formylated peptides.

### Example 8

### Demonstration that the System Works in vivo with Multiple Successive Epitopes

One major concern with any epitope delivery system is that, potentially, it might be effectively used only once due to the immune response to the carrier. Here we show that multiple injections are possible with our LFn delivery system. In this study, mice are initially injected with LFn-LCMV (H2^{b}) plus PA. Four weeks following this injection the mice are injected with LFn-LL₉₁₋₉₉ plus PA. Two weeks later the mice are assayed for a CTL response to LLO₉₁₋₉₉. We find that the mice mounted a strong LLO₉₁₋₉₉ CTL response and were not hindered by the initial injection. Other combinations may be tested using this methodology.

### Example 9

### The Use of an Epitope Containing a Single Mutation to Demonstrate the Overall Specificity of this System

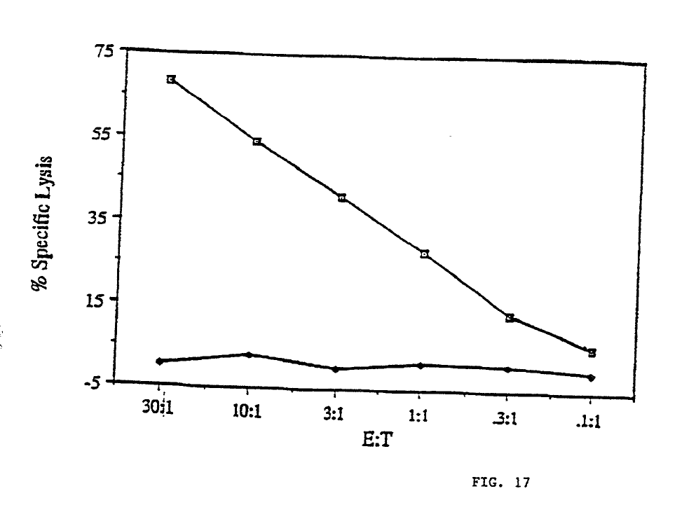

To show just how specific this system is, we investigated the ability of a CTL epitope containing a single mutation to stimulate a peptide specific CTL response. When a single amino acid in the LCN₁₁₈₋₁₂₆ epitope is changed (Q to E) all CTL stimulating activity is lost. This result further confirms peptide specificity of this response and that neither LFn nor PA are stimulating any response independent of the CTL peptide. These results are shown in FIG. 16.

### Example 10

### Capacity of the System to Provide Protection for Over Six Months After Initial Vaccination

We have found that mice vaccinated with LFn-LLO₉₁₋₉₉ are protected against L. monocytogenes at least up to 6 months following the injection.

### Example 11

### Construction of Reporter Fusion Proteins Having Affinity Handles

For the fusion constructs generated in these examples, a synthetic gene encoding DTA, the catalytic domain of diphtheria toxin, was utilized. The sequences of amino-terminal fusion peptides used in these investigations are shown in FIG. 4. DTA is an especially suitable reporter molecule for studying translocation: when introduced into the cellular cytoplasm, DTA causes cell death by catalyzing the ADP-ribosylation of elongation factor-2 (EF-2), inactivating the factor and thereby halting protein synthesis. Because DTA alone has little effect on cells, except at very high concentrations, the ability of PA to mediate translocation into the cytoplasm was tested by measuring the ability of DTA-fusion proteins to inhibit protein synthesis.

The hexa-histidine-DTA fusion protein (His-6-DTA), used in the initial experiments was expressed as a recombinant protein in E. coli and initially purified by nickel-chelate affinity chromatography. Subsequent anion-exchange FPLC resulted in purification of His-6-DTA to homogeneity. We obtained DTA by proteolytic cleavage of the fusion peptide at a modified thrombin recognition site, engineered to yield the amino-terminal sequence of authentic DTA. A final passage over a Ni²⁺-column resolved DTA from the fusion peptide and undigested His-6-DTA. Purified His-6-DTA, as well as all the fusion proteins constructed, cross-reacted with antisera to DT, and demonstrated similar ADP-ribosylation activity to DTA. We performed cytotoxicity experiments of all other DTA-fusion proteins in desalted cell lysates.

### Example 12

### Delivery of DTA Using Hexahistidine Affinity Handle

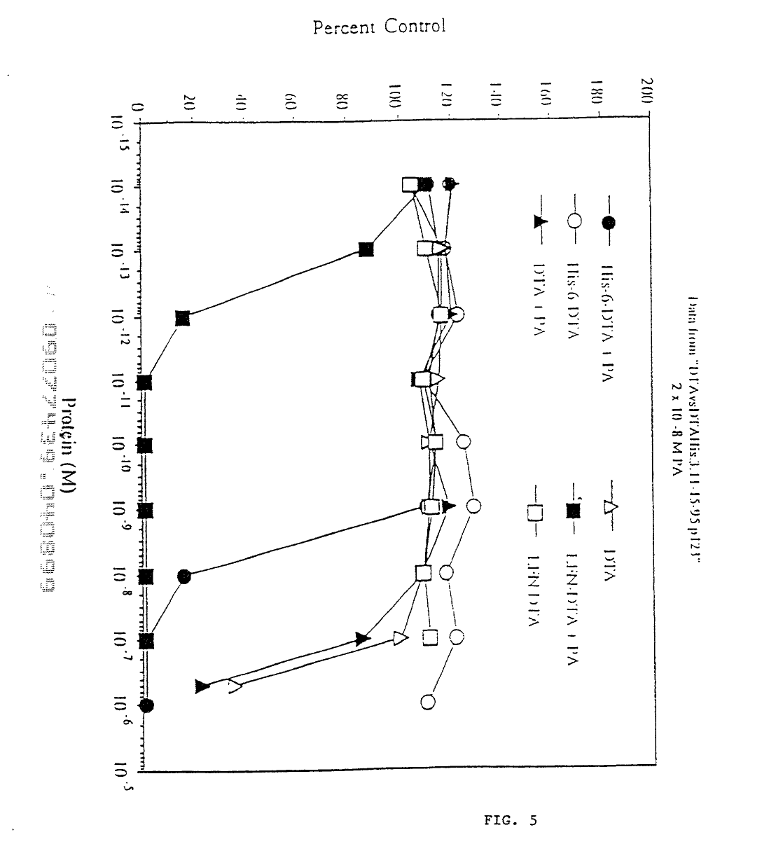

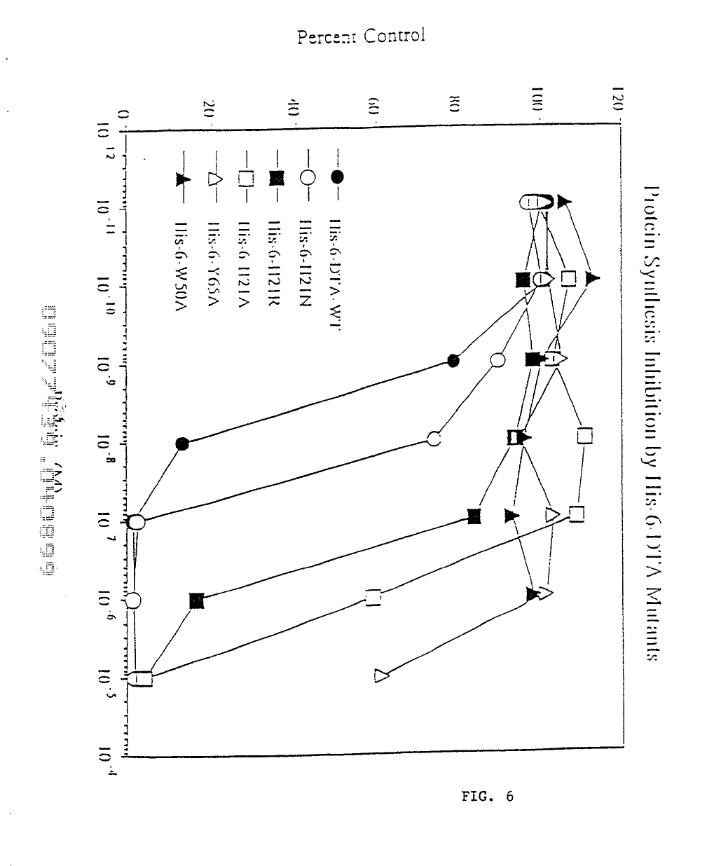

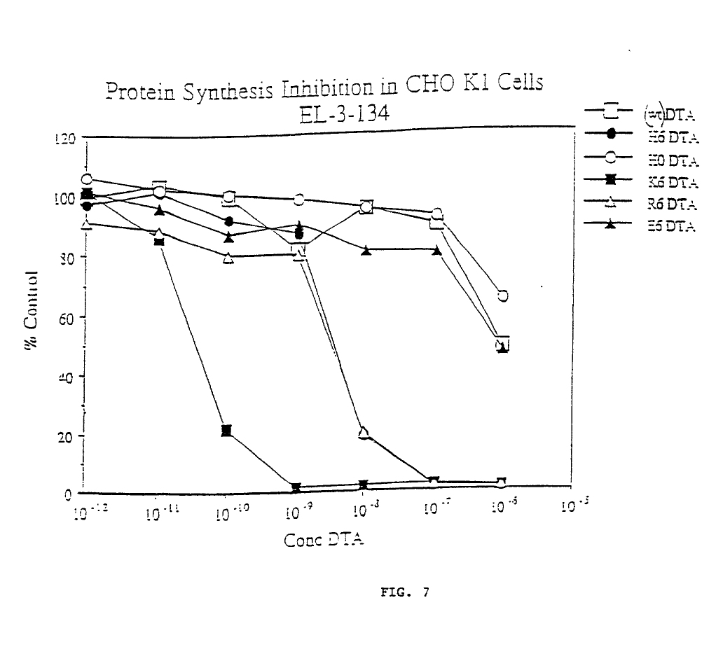

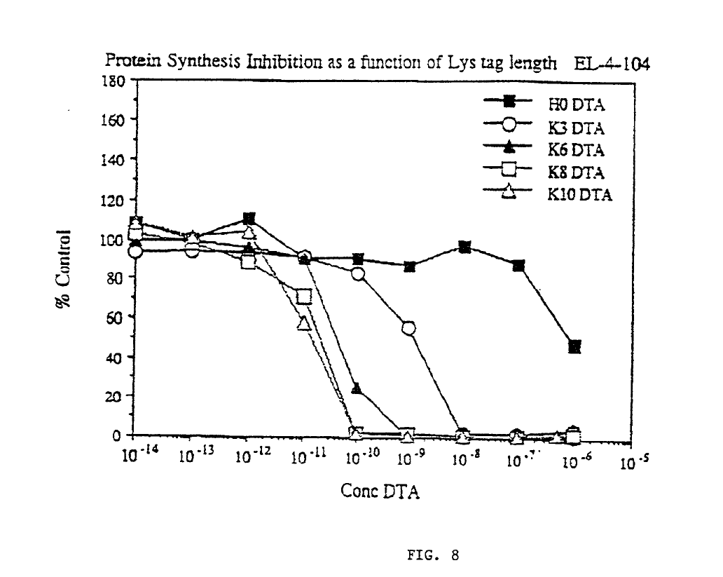

In the presence of PA, His-6-DTA was 100-fold more cytotoxic than DTA (FIG. 5). Without PA, His-6-DTA was not toxic to CHO-K1 cells over the concentration range tested. In the presence or absence of PA, DTA exhibited a degree of cytotoxicity at the highest concentration tested (5×10⁻⁷ hs M). As a control, the LFN-DTA fusion protein exhibited the same titration curves in the presence or absence of PA as previously reported, with an EC50 value of 3×10⁻¹³M. In the absence of PA, it is not clear why DTA was more cytotoxic than His-6-DTA. One explanation could be a loss of His-6-DTA due to electrostatic interactions of the polybasic peptide of His-6-DTA with the exposed carboxylate groups on the surface of the tissue culture treated plates used in these assays.

### Example 13

### Active Site Mutations in DTA Confirm Toxicity is Due to Delivery of DTA to the Cytoplasm

To confirm that the PA-dependent inhibition of protein synthesis was due to ADP-ribosylation of EF-2, we introduced five active site mutants into the His-6-DTA construct. These mutations—H21N, H21R, H21A, Y65A, and W50A—reduce ADP-ribosyltransferase activity in vitro by 3-, 70-, 120-, 670-, and 200,000-fold, respectively. The pattern of cytotoxicity reduction correlated well with the reductions in ADP-ribosyltransferase activity in vitro (FIG. 6).

These results indicate that the polyhistidine fusion peptide enhanced PA-mediated entry of DTA into the cytoplasm of CHO-K1 cells.

### Example 14

### Other Cationic Tags Function as PA-Dependent Affinity Handles

We investigated whether the six histidine residues were responsible for enhanced PA-mediated translocation by substituting these residues with lysines (Lys-6-DTA), arginines (Arg-6-DTA), and gluta-mates (Glu-6-DTA). As a control, we substituted the neutral sequence Ser-Ser-Gly-Ser-Ser-Gly (SSGSSG-DTA) for the six histidines (FIG. 4). When assayed for the ability to inhibit protein synthesis in the presence of PA, we found that Lys-6-DTA was a hundred-fold more cytotoxic than His-6-DTA (FIG. 7), while Arg-6-DTA exhibited cytotoxicity similar to His-6-DTA. In the presence of PA, SSGSSG-DTA and Glu-6-DTA were not cytotoxic to CHO-K1 cells (FIG. 7). These results show that 6 residues with chemically basic side-chains in the fusion peptide are sufficient for PA mediated translocation of DTA, and primary amines (Lys) are more effective in this system than imidazole (His) or guanidium (Arg) side chains. Furthermore, neutral or acidic-tagged peptides do not enhance PA-mediated translocation of DTA to the cytoplasm.

It was not initially clear why Lys-6-DTA was more cytotoxic than Arg-6-DTA. In later experiments, however, incubation with CHO-K1 cells for 24 hour at 37° C. resulted in Arg-6-DTA being extensively converted to a lower molecular weight species resembling DTA by SDS-PAGE analysis. Similar incubations with His-6-DTA and Lys-6-DTA resulted in no detectable degradation. These results indicate that the arginine residues of Arg-6-DTA may be susceptible to proteolytic degradation at the cell surface.

### Example 15

### Testing of Affinity Handles having Three, Eight, Ten, or Twelve Lysines

Because lysine residues were the most effective in enhancing PA-mediated translocation of DTA into CHO-K1 cells, we conducted subsequent experiments with lysine-tagged DTA fusion proteins. For Lys-6-DTA, we developed a convenient two-step purification protocol. The first step was designed to exploit the localized positive charge of the amino-terminal peptide; crude extracts were resolved using gravity-flow cation exchange chromatography (Whatman P-11 resin, Hillsboro, Oreg.). As a second step, Mono-Q™ anion exchange chromatography resulted in purification of Lys-6-DTA to homogeneity.

We substituted 3, 8, 10 or 12 lysines in the amino terminal fusion peptide to investigate how the number of positively charged residues affects PA-mediated DTA translocation (FIG. 4C). Lys-3-DTA, Lys-8-DTA, Lys-10-DTA, and Lys-12-DTA were prepared in crude lysates, and tested for their abilities to inhibit protein synthesis relative to Lys-6-DTA (FIG. 8). The number of lysine residues in the affinity handle directly correlated with the inhibition of protein synthesis observed. We believe that increasing numbers of Lysine residues in the affinity handle increase the overall affinity for the cell-surface or PA63. However, there may be a practical upper limit to the number of Lys residues that can be genetically engineered into the affinity handle. This is easily tested using the materials provided herein.

We have observed that a Lys-12-DTA fusion protein expressed poorly in E. coli and appeared to be susceptible to degradation. While these results do not establish the Lys-10 affinity handle as the upper limit in delivery efficiency, the ease of expression and purification of the Lys-10 tagged protein suggest that it may be the more practical of the two constructs.

### Example 16

### Affinity Handle Delivery is Independent of LFn on PA Binding Sites

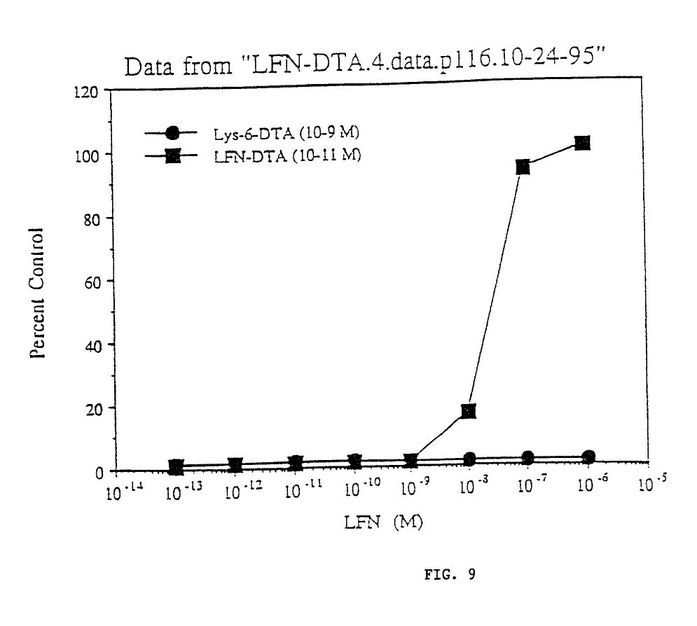

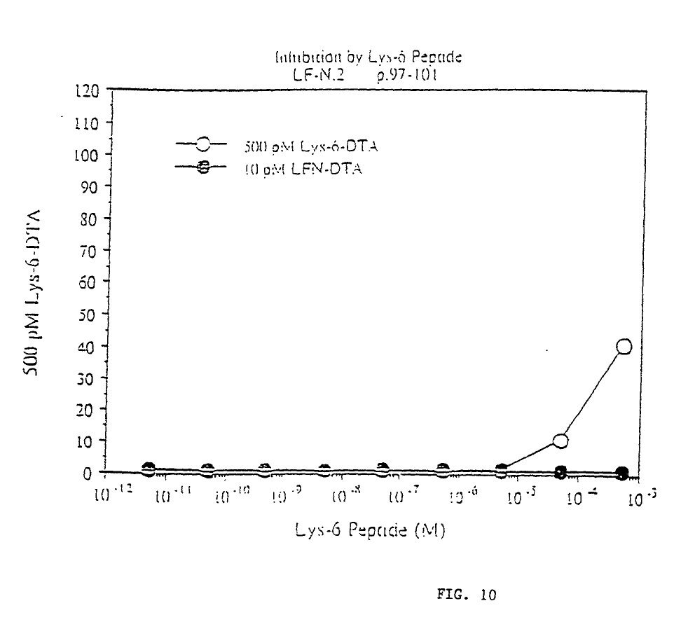

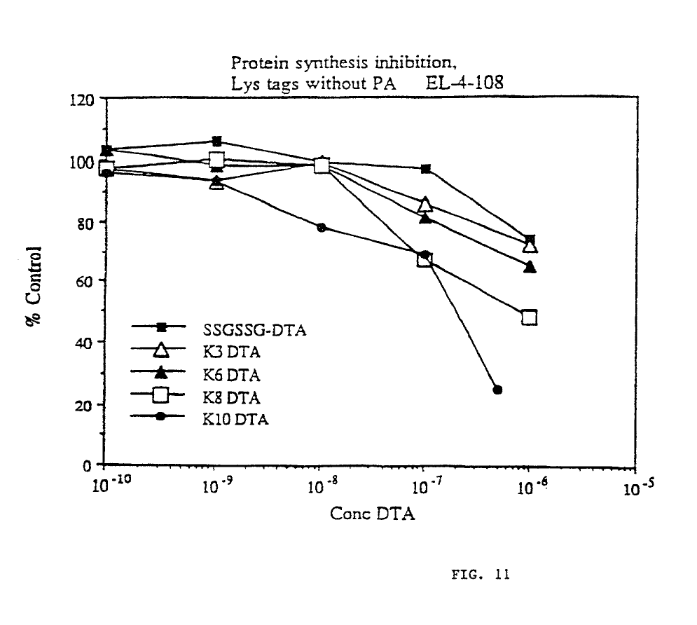

A question that arises from this work is whether these polybasic fusion peptides bind directly to PA or to another component at the cell surface. If Lys-6-DTA binds to the same site on PA as LF, then LFN should block cytotoxicity by competing for PA. As reported previously, LFN does protect cells from the LFN-DTA fusion protein in a dose-dependent manner. However, as shown in FIG. 9, when incubated with CHO-K1 cells in the presence of PA and Lys-6-DTA, LFN does not protect cells from the cytotoxic effects of the fusion protein, even at 1000-fold molar excess of LFN. In related experiments, a synthetic peptide with the sequence KKKKKKGSGCG did not protect CHO-K1 cells from the PA-dependent cytotoxic effects of LFN-DTA, even at 5×10⁷ molar excess (FIG. 10). The same peptide was able to slightly protect CHO-K1 cells from the PA-dependent cytotoxic effects of Lys-6-DTA, but only at 100,000 or greater molar excess, suggesting either a very high number of Lys-6-DTA binding sites, or intrinsically less binding affinity of the synthetic peptide to the Lys-6-DTA binding site. Collectively, these results indicate that Lys-6-DTA and LFN do not share identical PA binding sites.

### Example 17

### Interchangeable Nature of Heterologous Protein

Exoenzyme S (from Pseudomonas aeruginosa) may be used to address the generality of the PA-mediated translocation system we provide. Initial experiments have indicated that exoenzyme S, for which there is no evidence for cellular entry absent an affinity handle, is able to enter cells when tagged with an amino-terminal polyhistidine fusion protein in the presence of PA. His-6 tagged exoenzyme S enters cells, as measured by decreased cell viability, in the presence of PA.

### Example 18

### PA-Independent Compound Delivery Using Cationic Affinity Handles

We have obtained data indicating that basic residues in the affinity handles are able to mediate entry of DTA into the cytoplasm of cells, even in the absence of PA (FIG. 11). Protein synthesis inhibition was seen in the absence of PA at concentrations of lysine affinity handle—DTA which approached the highest employed in normal protein synthesis inhibition experiments (i.e. 5e⁻⁷ M-1e⁻⁶ M); this inhibition of protein synthesis became more pronounced as the number of lysines in the affinity handle increased. In particular, 5e⁻7 M Lys-10-DTA lowered protein synthesis to 25% of control. Although the mechanism by which such affinity handle-DTA apparently enters cells in the absence of PA is not known, we speculate that it is related to the presumed increase in affinity of the handles for a cell surface component or PA.

### Example 19

### Use of Polycationic Affinity Handles in vivo

In this example we test the polycationic tag approach in vivo. As a simple reporter system we use the enzymatic domain of diphtheria toxin (DTA) fused to 6 lysines. Mice are injected with Lys-DTA plus PA and delivery is assayed by death of the animal. We find that mice injected with Lys-6-DTA plus PA do not survive. Mice injected with Lys-DTA without PA or DTA plus PA are not affected. These results indicate that the DTA delivery is PA-mediated and Lys tag-dependent. Our results indicate that the polycationic handle system has potential for in vivo delivery of heterologous molecules.

Other embodiments are within the following claims.

What we claim is:

1. A method of introducing an antigenic compound into the cytoplasm of a cell, said method comprising contacting the cell with a fusion molecule, wherein said fusion molecule comprises a delivery molecule selected from a group consisting of a toxin delivery molecule and a polycationic affinity handle, said delivery molecule linked to the antigenic compound.

2. A method of introducing a nucleic acid encoding an antigenic compound into the cytoplasm of a cell, said method comprising contacting the cell with a fusion molecule, wherein said fusion molecule comprises a delivery molecule selected from a group consisting of a toxin delivery molecule and a polycationic affinity handle, and wherein said delivery molecule is linked to said nucleic acid.

3. The method of claim 1 or 2, further comprising contacting the cell with a B moiety of a toxin.

4. The method of claim 1 or 2, wherein said toxin delivery molecule is LFn of anthrax.

5. The method of claim 3, wherein said B moiety is anthrax PA.

6. The method of claim 5, wherein said anthrax PA is the 63 kDa carboxy-terminal domain of anthrax PA.

7. The method of claim 1 or 2, wherein said polycationic affinity handle comprises a peptide of 2 to 250 amino acid residues.

8. The method of claim 7, wherein said polycationic affinity handle comprises a peptide of 2 to 16 amino acid residues.

9. The method of claim 7, wherein at least two of the amino acids of said peptide are selected from the group consisting of: arginine, lysine, and histidine, and wherein at least 10% of the amino acids comprising said peptide are selected from the group consisting of: arginine, lysine, and histidine.

10. The method of claim 9, wherein said handle comprises at least 3 amino acid residues selected from the group consisting of arginine, lysine, and histidine.

11. The method of claim 10, wherein said handle comprises at least 6 arginine residues.

12. The method of claim 9, wherein said handle comprises at least 3 lysine residues.

13. The method of claim 9, wherein said handle comprises at least 6 histidine residues.

14. The method of claim 1 or 2, wherein the pKₐ of the polycationic affinity handle is between 6.5 and 12.5.

15. The method of claim 1 or claim 2, wherein the antigenic compound is selected from a group consisting of: Human Papillomavirus 16 peptides (e.g., antigens E6 and E7, E7 peptide 49-57 RAHYNIVTF); human P53 peptides (e.g., V10 peptide FYQLAKTCPV); human immunodeficiency virus peptides (e.g., gp 120, P18 peptide RIQRGPGRAFVTIGK); MUC-I human cancer antigen peptides; peptides from proteins of MAGE gene family (e.g., MAGE-1 SAYGEPRKL, MAGE-3 FLWGPRALV); peptides from the human tyrosinase protein (e.g., Tyr-A2-1 MLLAVLYCL, Try-A@-2 YMNGTMSQV); Listeriolysin-O peptides (e.g., LLO91-99 GYKDGNEYI); P60 peptides (e.g., P60217-225 KYGVSVQDI); MART-1 peptides (e.g., M-9 AAAAAGIGILTV, M10-3 EAAGIGILTV); BAGE-1 peptides (e.g., AARAVFLAL); P1A peptides (e.g., P815A35-43 LPYLGWLVF); Connexin gap junction derived peptides (e.g., Mut 1 FEQNTAQP, MUT 2 FEQNTAQA); peptides/proteins from any of the following pathogens: Cytomegalovirus, Hepatitis B, Human Herpes Virus 1-5, Rabies Virus, Meassles Virus, Mumps Virus, Rubella Virus, Shigella, Mycobacterium tuberculosis and avium, Salmonella typhi and typhimurium, HTLV-I,II, Varicella zoster, Variola, Polio, Yellow Fever, Encephalitis viruses, and Epstein-Barr virus.

16. The method of claim 2, wherein the nucleic acid is selected from a group consisting of DNA and RNA.

17. The method of claim 9, wherein said fusion molecule further comprises a peptide bond linking said delivery molecule to said compound.

18. The method of claim 17, wherein said peptide bond is at the amino-terminus of said delivery molecule.

19. The method of claim 17, wherein said peptide bond is at the carboxy-terminus of said delivery molecule.

20. The method of claim 1 or 2, wherein said fusion molecule further comprises an amide bond linking said delivery molecule to said compound.

21. The method of claim 1 or 2, wherein said fusion molecule further comprises a thioether bond linking said delivery molecule to said compound.

22. The method of claim 1 or 2, wherein said fusion molecule further comprises a disulfide bond linking said delivery molecule to said compound.

23. The method of claim 1 or 2, wherein said fusion molecule further comprises a cleavage site between said compound and said polycationic affinity handle.

24. The method of claim 1 or 2, wherein said fusion molecule further comprises a spacer between said compound and said delivery molecule.

25. A kit for introducing a antigenic compound into the cytoplasm of a cell, said kit comprising a fusion molecule, wherein said fusion molecule comprises a polycationic affinity handle or a toxin delivery molecule linked to said compound.

26. A kit for introducing a compound into the cytoplasm of a cell, said kit comprising a fusion molecule, wherein said fusion molecule comprises a polycationic affinity handle linked to said compound.

27. The kit of claim 25 or 26, wherein said kit further comprises a B moiety of a toxin.

28. The kit of claim 27, wherein said B moiety is anthrax PA.

29. A fusion molecule for delivery of an antigenic compound to the cytoplasm of a cell, said fusion molecule comprising said antigenic compound linked by a covalent bond to a second compound, said second compound being a polypeptide, said polypeptide being selected from the group consisting of a polycationic affinity handle, LFN, or a toxin molecule related to LFN.

30. The fusion molecule of claim 29, wherein said fusion molecule has at least two antigenic compounds covalently linked to said second compound.

31. A fusion molecule for delivery of an nucleic acid to the cytoplasm of a cell, said fusion molecule comprising said nucleic acid linked by a covalent bond to a second compound, said second compound being a polypeptide, said polypeptide being selected from the group consisting of a polycationic affinity handle, LEN, or a toxin molecule related to LFN.

32. The fusion molecule of claim 29, wherein said antigenic compound is selected from the group consisting of: Human Papillomavirus 16 peptides (e.g., antigens E6 and E7, E7 peptide 49-57 RAHYNIVTF); human P53 peptides (e.g., V10 peptide FYQLAKTCPV); human immunodeficiency virus peptides (e.g., gp 120, P18 peptide RIQRGPGRAFVTIGK); MUC-I human cancer antigen peptides; peptides from proteins of MAGE gene family (e.g., MAGE-1 SAYGEPRKL, MAGE-3 FLWGPRALV); peptides from the human tyrosinase protein (e.g., Tyr-A2-1 MLLAVLYCL, Try-A@-2 YMNGTMSQV); Listeriolysin-O peptides (e.g., LLO91-99 GYKDGNEYI); P60 peptides (e.g., P60217-225 KYGVSVQDI); MART-1 peptides (e.g., M-9 AAAAAGIGILTV, M10-3 EAAGIGILTV); BAGE-1 peptides (e.g., AARAVFLAL); P1A peptides (e.g., P815A3543 LPYLGWLVF); Connexin gap junction derived peptides (e.g., Mut 1 FEQNTAQP, MUT 2 FEQNTAQA); peptides/proteins from any of the following pathogens: Cytomegalovirus, Hepatitis B, Human Herpes Virus 1-5, Rabies Virus, Meassles Virus, Mumps Virus, Rubella Virus, Shigella, Mycobactenum tuberculosis and avium, Salmonella typhi and typhimunium, HTLV-I,II, Varicella zoster, Variola, Polio, Yellow Fever, Encephalitis viruses, and Epstein-Barre virus.

33. The fusion molecule of claim 30, wherein said antigenic compound is selected from the group consisting of DNA and RNA.

34. A composition comprising a mixture of a fusion molecule and the B moiety of a toxin, said fusion molecule comprising an antigenic compound linked by a covalent bond to a second compound, said second compound being a polypeptide, said polypeptide being a polycationic affinity handle, LFN, or a polypeptide from a toxin delivery system related to LFN.

35. A composition comprising a mixture of a fusion molecule and the B moiety of a toxin, said fusion molecule comprising a nucleic acid linked by a covalent bond to a second compound, said second compound being a polypeptide, said polypeptide being a polycationic affinity handle, LFN, or a polypeptide from a toxin delivery system analogous to LFN.

36. A method of introducing a fusion molecule into the cytoplasm of a cell, comprising contacting the cell with said fusion molecule, wherein said fusion molecule comprises a polycationic affinity handle covalently linked to a compound.

37. The method of claim 1, further comprising contacting the cell with a B moiety of a toxin.

38. The method of claim 2, wherein said B moiety is anthrax PA.

39. The method of claim 3, wherein said anthrax PA is the 63kEa carboxy-terminal domain of anthrax PA.

40. The method of claim 1, wherein said polycationic affinity handle comprises a peptide of 2 to 250 amino acid residues.

41. The method of claim 5, wherein said polycationic affinity handle comprises a peptide of 2 to 16 amino acid residues.

42. The method of claim 5, wherein at least two of the amino acids of said peptide are selected from the group consisting of: arginine, lysine, and histidine, and wherein at least 10% of the amino acids comprising said peptide are selected from the group consisting of: arginine, lysine, and histidine.

43. The method of claim 7, wherein said handle comprises at least 3 amino acid residues selected from the group consisting of arginine, lysine, and histidine.

44. The method of claim 8, wherein said handle comprises least 6 arginine residues.

45. The method of claim 8, wherein said handle comprises at least 3 lysine residues.

46. The method of claim 8, wherein said handle comprises at least 6 histidine residues.

47. The method of claim 1, wherein the pKₐ of the polycationic affinity handle is between 6.5 and 12.5.

48. The method of claim 1, wherein the compound is selected from a group consisting of: a protein toxin molecule, an apoptosis inducing molecule, a protein component of the signal transduction pathway, DNA, RNA, an MHC class I antigens, a protein for genetic complementation, a therapeutic peptide and a therapeutic protein.

49. The method of claim 7, wherein said fusion molecule further comprises a peptide bond linking said polycationic affinity handle to said compound.

50. The method of claim 7, wherein said peptide bond is at the amino-terminus of said polycationic affinity handle.

51. The method of claim 7, wherein said peptide bond is at the carboxy-terminus of said polycationic affinity handle.

52. The method of claim 1, wherein said fusion molecule further comprises an amide bond linking said polycationic affinity handle to said compound.

53. The method of claim 1, wherein said fusion molecule further comprises a thioether bond linking said polycationic affinity handle to said compound.

54. The method of claim 1, wherein said fusion molecule further comprises a disulfide bond linking said polycationic affinity handle to said compound.

55. The method of claim 1, wherein said fusion molecule further comprises a cleavage site between said compound and said polycationic affinity handle.

56. The method of claim 1, wherein said fusion molecule further comprises a spacer between said compound and said polycationic affinity handle.

57. A kit for introducing a compound into the cytoplasm of a cell, said kit comprising a fusion molecule, wherein said fusion molecule comprises a polycationic affinity handle linked to said compound.

58. The kit of claim 22, wherein said kit further comprises a B moiety of a toxin.

59. The kit of claim 23, wherein said B moiety is anthrax PA.

60. A fusion molecule for delivery of a first polypeptide to the cytoplasm of a cell, said fusion molecule comprising said first polypeptide linked by a covalent bond to a second polypeptide, said second polypeptide having at least two amino acids, at least two of said amino acids being selected from the group consisting of arginine, lysine, and histidine, wherein if the covalent bond is a peptide bond at least one of said amino acids is arginine or lysine.

61. The fusion molecule of claim 25, wherein said first polypeptide is selected from the group consisting of protein toxin molecules, apoptosis inducing molecules, protein components of the signal transduction pathway, DNA, RNA, MHC class I antigens, proteins for genetic complementation, therapeutic peptides and therapeutic proteins.

62. A composition comprising a mixture of a fusion molecule and the B moiety of a toxin, said fusion molecule comprising a first peptide linked by a covalent bond to a second polypeptide, said second peptide having at least 3 amino acids, at least 3 of said amino acids being selected from the group consisting of arginine, lysine, and histidine, wherein if said covalent bond is a peptide bond at least one of said amino acids of said second peptide is arginine or lysine.

63. A method of introducing a nucleic acid into the intracellular region of a cell, comprising contacting the cell with (a) said nucleic acid and (b) a polycationic affinity handle, wherein polycationic affinity handle is brought into close proximity of said nucleic acid by electrostatic forces.
